(19) United States
(12) Patent Application Publication
(10) Pub. No.: US 2022/0008354 A1
(43) Pub. Date: Jan. 13, 2022

(54) ORALLY ADMINISTRABLE CANNABINOIDS-CONTAINING COMPOSITIONS AND METHODS

(71) Applicant: YISSUM RESEARCH DEVELOPMENT COMPANY OF THE HEBREW UNIVERSITY OF JERUSALEM LTD., Jerusalem (IL)

(72) Inventors: Elka TOUITOU, Hod Hasharon (IL); Hiba NATSHEH, Jerusalem (IL)

(21) Appl. No.: 17/289,932

(22) PCT Filed: Oct. 31, 2019
(86) PCT No.: PCT/IL2019/051180

§ 371 Date: Apr. 29, 2021

Related U.S. Application Data
(60) Provisional application No. 62/755,483, filed on Nov. 4, 2018.

(51) Int. Cl. A61K 31/05; A61K 9/00; A61K 47/24; A61K 9/48; A61K 31/352

(52) U.S. Cl. CPC A61K 31/05; A61K 9/0053; A61K 31/352; A61K 9/4858; A61K 9/4866; A61K 47/24

(57) ABSTRACT

Essentially water-free liquid composition for oral administration, comprising: from 25% to 75% by weight of one or more cannabinoid (s); and from 25% to 59% by weight one or more phospholipid (s); and optionally one or more antioxidants (s).

It has long been recognized that the active ingredients of cannabis are able to provide relief for a variety of symptoms and conditions, for example, to reduce pain. The major components include cannabidiol (CBD), tetrahydrocannabinol (THC) and cannabinol (CBN). The term “cannabinoid”, as used herein, is meant to include compounds interacting with cannabinoid receptors, either naturally occurring or synthetic compounds, e.g., each of the aforementioned components, derivatives and analogues thereof, as described further below.

Cannabinoids are generally difficult to formulate, e.g., into pharmaceutical dosage forms, due to their strong lipophilic character, indicated by their high log P values (octanol/water partition).

A novel approach towards orally administrable cannabinoids formulations was recently presented in WO 2017/098502, where it was shown that mixtures consisting of cannabinoids and preferably not less 60% by weight phospholipids create compact masses that can be easily processed and shaped into dosage forms suitable for oral delivery. In the formulations tested in WO 2017/098502, phospholipids generally constitute the major component; for instance, in Example 6 of WO 2017/098502, it is reported that solid compositions consisting of cannabinoids and phospholipids at weight ratios of 1:9, 3:7 and 4:6 were subjected to disintegration tests in simulated gastric fluid (2 hours) and then in simulated intestinal fluid (24 hours). The solid formulations of WO 2017/098502 did not disintegrate during the test period.

There exists a need for orally administrable, cannabinoids-rich liquids or viscous liquids, that can be put inside gelatin capsules to serve various therapeutic goals, for example, rapid relief of pain.

We have now found that suitably proportioned mixtures of cannabinoids and phospholipids create non-solid compositions (i.e., liquids, viscous liquids), with high cannabinoids concentration (i.e., not less than 25% based on the total weight of the preparation). In general, the cannabinoid(s) constitute the predominate component of the composition, i.e., the concentration of the cannabinoids is higher than that of the phospholipids. But in some of the compositions of the invention, the combination consisting of cannabinoids/phospholipids is approximately equally proportioned, at a weight ratio in the range from 4:3 to 3:4 (that is, 1:0.75-1.33).

Accordingly, the invention is primarily directed to a liquid composition for oral administration, comprising:

not less than 25% by weight of one or more cannabinoid(s), e.g., from 25% to 75%; and
from 25% to 59% by weight one or more phospholipid(s), e.g., up to 58%, 57%, 56% or up to 55%.

Concentrations reported herein are by weight percentage based on the total weight of the composition, unless indicated otherwise.

The composition preferably comprises one or more antioxidant (s).

The compositions of the invention are “non-aqueous”, namely, are essentially water-free (i.e., containing less than 10 wt %, less than 5 wt %, less than 1 wt %, less than 0.5 wt % water, especially water-free (0% water)). Compositions comprising from 25 to 70%, e.g., from 25 to 55%, more specifically 30% to 50% by weight of one or more cannabinoid(s) and from 30% to 50% by weight of one or more phospholipid(s) are preferred.

The cannabinoid/phospholipids mixtures can be obtained in a liquid (e.g., viscous liquid) form upon mixing the two solid components, when the mixing takes place at room temperature. Another aspect of the invention is an orally administrable liquid composition obtainable by, or obtained by, mixing cannabinoid(s) and phospholipids to form a liquid, characterized in that the mixing takes place in the absence of other ingredients, e.g., at room temperature. That is, addition of solvents/diluents may follow, but only after the cannabinoid(s) and phospholipids are associated in a liquid form. The composition is then loaded into a capsule, which forms another aspect of the invention.

It should be noted that cannabinoid(s) and phospholipids are solids at room temperature, but owing to their ability to form a liquid when mixed under the conditions described herein, without the aid of solvents/diluents, it is possible to formulate the cannabinoids into highly concentrated liquids. As indicated by the experimental results reported below, cannabinoid(s) can be formulated into >50% weight percent cannabinoid(s)-containing liquids which are free of solvents/diluents. Such highly rich cannabinoid(s) liquids form specific aspect of the invention (wherein the concentration of the cannabinoid(s) is higher than 45%, higher than 50%, higher than 60%, e.g., up to 70%-75% by weight).

As pointed out above, some compositions of the invention are based on roughly equally proportioned mixtures of cannabinoid(s) to phospholipid(s). By “roughly equally proportioned mixtures” are meant mixtures where the weight ratio cannabinoid(s) to phospholipid(s) is in the range from 4:3 to 3:4 (1:0.75-1.33), e.g., from 5:4 to 4:5 (1:0.8-1.25, for example, about 1:1. Such equally proportioned compositions of the invention will generally include one or more solvents as described below.

The liquid compositions of the invention can be encapsulated in capsules, e.g., gelatin capsules to provide liquid-filled capsule.

To prepare the compositions of the invention, the cannabinoid compounds, either natural or synthetic, may be utilized in a solid form (for example, an isolated synthetic compound that underwent purification by crystallization), or in the form of an extraction concentrate, solvent extract, oil extract and oil solution, possibly surfactant-containing extracts and solutions. A non-limiting list of cannabinoids is given below:

CBD (chemical named 2-[3-methyl-6-(1-methylethenyl)-2-cyclohexen-1-yl]-5-pentyl-1,3-benzenedi-ol). The synthesis of CBD was described, for example, by Gaoni Y, Mechoulam R [Tetrahedron Letters. 26 (8): 1083-1086 (1985)]; and by Petilka et al. [Helv. Chim. Acta, 52:1102 (1969); and in J. Am. Chem. Soc., 87:3273 (1965)]. Δ⁹-THC, available under the name dronabinol; and Δ⁸-THC.

CBN (chemically named 6,6,9-trimethyl-3-pentyl-6H-dibenzo[b,d]pyran-1-ol). The synthesis of CBN was described by Novak et al., Tetrahedron Letters, 23:253 (1982); and by Jesse A. Teske and Alexander Deiters Org. Lett., 2008, 10 (11), pp 2195-2198.

Nabilone (chemically named: 3-(1,1-dimethylheptyl)-6,6a,7,8,10,10a-hexahydro-1-hydroxy-6,6-dimethyl-9-H-dibenzo[b,d]pyran-9-one). The preparation of this synthetic cannabinoid is described, for example, in U.S. Pat. No. 3,968,125.

Levonantradol (chemically named: (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate. The preparation of this synthetic cannabinoid is described, for example, in U.S. Pat. Nos. 4,206,225, 4,232,018, 4,260,764, 4,235,913, 4,243,674, 4,263,438, 4,270,005, and 4,283,569.

(−)-HU-210 (chemically named: (−)-(3S,4S)-7-hydroxy-Δ⁶-tetrahydrocannabinol-1,1-dimethylhept-yl). The preparation of this synthetic cannabinoid can be found in U.S. Pat. Nos. 4,876,276 and 5,521,215.

(+)-HU-210 (chemically named: (+)-(3S,4S)-7-hydroxy-Δ⁶-tetrahydrocannabinol-1,1-dimethylhept-yl). The preparation of this synthetic cannabinoid is described in U.S. Pat. Nos. 4,876,276 and 5,521,215.

11-hydroxy-Δ⁹-THC, which can be prepared via the synthetic route described by Siegel et al., J. Org. Chem., 54:5428 (1989).

Δ⁸-tetrahydrocannabinol-11-oic acid, which is naturally occurring derivative and can be produced synthetically employing methods described in U.S. Pat. No. 6,162,829.

CP 55,940 (chemically named: 4-(1,1-dimethylheptyl)-2,3′ dihydroxy-6′alpha-(3-hydroxypropyl)-1′,2′,3′,4′,5′,6′-hexahydrobiphenyl), which is commercially available from Tocris Cookson, Inc., Its preparation has been described; see for example U.S. Pat. Nos. 4,371,720 and 4,663,474.

R(+)-WIN 55,212-2 (chemically named: (R)-(+)-[2,3-dihydro-5-methyl-3-(4-morpholinylmethyl)-pyrrolo[1,2,3-de]-1-,4-benzoxazin-6-yl]-1-naphthalenyl-methanone) is commercially available in the form of its mesylate salt from various manufacturers.

It should be noted that the compounds listed above may be used in the form of pharmaceutically acceptable salts or metabolic precursors (e.g., prodrugs that are metabolized in the patient's body as described in U.S. Pat. No. 5,847,128). Crude herbal cannabis—in countries and jurisdictions where it is, or will become, legally allowed—can also be delivered using the composition of this invention. The term “cannabinoid”, as used herein, includes cannabinoid acids.

The preferred cannabinoids are selected from the group consisting of CBD, THC, CBN, and mixtures thereof.

Turning now to the phospholipids, they are preferably present in the compositions of the invention at a concentration in the range from 25 to 55%, preferably from 30 to 50% by weight based on the total weight of the composition, more specifically from 30 to 45% by weight, e.g., from 30 to 40%. Phospholipids suitable for use in the preparation of the composition according to the present invention include phosphoglycerides, e.g., phosphatidylcholine (lecithin, such as soy, sunflower, and egg lecithin). Other phospholipids can be selected from hydrogenated phosphatidylcholine, phosphatidylserine, phosphatidylethanolamine, phosphatidylglycerol, phosphatidylinositol and mixtures thereof. Phosphatidylcholine is preferred; suitable phosphatidylcholine products are commercially available from various sources, for example, from Lipoid under the brand names of Phospholipon®: the 85 G, 90G and 80 H, 90H grades and their mixtures; Lipoid®: Lipoid 100S PC, Lipoid S 100, Lipoid S 75 or from Perimondo under the brand names of Sunlipon®: Sunlipon®90, Sunlipon® 65, Sunlipon® 50, their mixtures and others.

Antioxidants are present in the compositions of the invention, e.g., at a concentration from 0.05 to 2% by weight based on the total weight of the composition. Suitable antioxidants include tocopherols and tocopherol derivatives (vitamin E), 3,5-Di-tert-4-butylhydroxytoluene (BHT), butylated hydroxyanizole (BHA), vitamin C, sodium metabisulfite, potassium metabisulfite, ascorbic acid, lycopene, ascorbyl palmitate and the like. Mixtures of antioxidants may be used.

As shown below, the liquid cannabinoids/phospholipids mixture can either be devoid of auxiliary liquids, or can be combined with liquids such as glycols and vegetable oils.

The glycol, when used, is a water-miscible diol such as propylene glycol. The glycol content of the composition is from 0.1% by weight based on the total weight of the composition, and up to about 40% by weight, more specifically, from 5 to 30% by weight. It should be noted that the composition of the invention is essentially water-free and in general is also devoid of (C2-C4) volatile mono-alcohols such as ethanol and isopropanol which are used in phospholipids-based vesicular preparations. That is, phospholipids are not arranged in a vesicular structure in the composition of the invention. However, small amounts of low alcohols can still be present in the composition, for example, each up to 5-10% by weight based on the total weight of the composition, as long as their presence does not cause the phospholipids to take-up a vesicular structure.

Suitable oils include black cumin seed oil, hemp seed oil, pomegranate seed oil, sesame seed oil, brassica seed oil and black sesame oil, to name a few [hemp seed oil is produced by cold pressing the seeds of the Cannabis sativa and should not be confused with extractable materials made from the cannabis flower and leaves. Hemp seed oil may be used in the present invention either in a crude form (protein-containing) or in a refined form, following removal of the proteins].

In some embodiments of the invention, the total concentration of the liquid component used in the preparation of the composition (the glycol(s), the vegetable oil(s) or a mixture thereof) is not less than 15% by weight, e.g., not less than 18% by weight.

When the oil is added as a sole liquid component of composition, its concentration may be from 18 to 50% by weight. When present in conjunction with glycol(s), the concentration of the vegetable oil in the composition is not less than 0.005% by weight, preferably from 0.02 to 15%, e.g., 0.5 to 10%, for example from 1 to 5% by weight.

Turning now to the method of preparation of the compositions of the invention, different techniques may be employed to produce the compositions of the invention consisting of the components listed above. One possible order of addition involves first mixing well the one or more phospholipids then adding with mixing the one or more cannabinoids and mixing to obtain a liquid composition, followed by addition of the antioxidant and/or the other components. On a laboratory scale, when the quantity of the composition is small, the composition may be mixed using, for example, mortar and pestle. On a larger scale, mixing is achieved using an acceptable instrument such as homogenizer or a mixer.

The invention also provides a unit dosage form filled with liquid composition of the invention. For example, two-parts capsule and capsules used for softgel (hard-shelled capsules, soft-shelled capsules). The unit dosage contains the liquid composition of the invention.

To provide dual release profile, the solid composition of WO 2017/098502 may be incorporated into the liquid-containing unit dosage form described above. Thus, another aspect of the invention is a unit dosage form comprising the cannabinoids-containing liquid described above and a cannabinoid(s)-containing solid consisting essentially of phospholipids/cannabinoids at weight proportion of at least 60:40 in favor of the phospholipids, preferably at least 70:30, e.g., at least 80:20. The preparation of suitably-shaped solid phospholipids/cannabinoids bodies is described in Example 5 of WO 2017/098502.

The cannabinoids-containing liquid and cannabinoids-containing solid can be combined in a single unit dosage form using various encapsulation approaches. The cannabinoids and phospholipids components of the liquid and solid formulations incorporated into the unit dosage forms may be the same or different.

It should be noted that the cannabinoids-containing solid may take the shape of a single mass or be provided as a plurality of small bodies.

According to a first approach, a single two-piece capsule is used. A composition according to WO 2017/098502 made of the phospholipids/cannabinoids >60:40 proportioned mixture, e.g., 70:30-90:10 mixtures, is suspended in the rapid release cannabinoids-containing liquid formulation of the present invention encapsulated in hard capsules, e.g., two-piece capsules made of gelatin or Hypromellose.

According to a second approach that is based on capsule-in-capsule technologies, such as those available in the marketplace, the invention provides a unit dosage form comprising an inner capsule (e.g. gelatin capsule) loaded with one or more of the cannabinoids-containing solid bodies according to WO 2017/098502, wherein said inner capsule is encapsulated in cannabinoids-liquid filled capsule of the liquid of the present invention.

A third approach is based on a single capsule divided into two separate spaces, each filled with the liquid and solid composition, respectively. For example, a hemisphere or half capsule shell is filled with the cannabinoids-containing liquid of the invention and sealed. The one or more cannabinoids-containing solid bodies is(are) placed in a second hemisphere or half capsule shell, which is sealed and joined to the liquid-containing part.

It should be noted that the composition of the invention is not limited to the delivery of cannabinoids as the sole active ingredient, namely, it may be used to provide combination therapy. That is, a second active ingredient could be added to the composition and unit dosage forms described herein, and administrated as described above and as illustrated below. In case of the dual unit dosage form specifically described above, one or more active ingredients may be added either to the liquid formulation, solid formulations or both.

Cannabinoids can be administered via the oral route with the aid of the composition of the invention to treat any disease or condition where cannabinoids could have impact, e.g., by combating the progress of the disease, or by relieving symptoms associated with the disease in a mammal (human, animal, pet). The following diseases and conditions that are treated by cannabinoids can be mentioned: neurological disorder, muscular disturbances, ticks, insomnia, pain, anxiety, migraine, glioma, epilepsy, blastoglioma, cancer, acne, IBD, Chron's disease, loss of appetite, anxiety, distress, panic, tremor, multiple sclerosis, menopause including symptoms associated with menopause such as hot flushes, autism, dementia, Alzheimer, Parkinson, awakens, mood disorders, post-trauma, alcoholic and nonalcoholic fatty liver, hysteria, seizure and types of encephalopathy, including hepatic-encephalopathy and other liver diseases such as hepatic cancer and cirrhosis, menstrual pain and cramps, premenstrual pain, painful menstrual periods and vaginal mucosa inflammation.

Hence, another aspect of the invention is a method of treatment, in particular treatment of illnesses and conditions set out above and/or symptoms associated therewith, which method comprises the oral administration to a mammal of a liquid composition comprising at least one cannabinoid, phospholipids, an antioxidant and optionally glycol, optionally a vegetable oil, as described above.

One specific aspect of the invention is a method for treating (relieving) pain, for example, in patients with neurological diseases, such as multiple sclerosis, LS, or chronic pain (e.g., pain associated with the nervous system), comprising the oral administration of the liquid composition of the invention.

Pharmaceutically active compounds can be added to the composition of the invention, such as analgesics (including opioid analgesics), sedative, anti-anxiety drugs and anticonvulsants, for example, tramadol HCl, diazepam, brotizolam and, melatonin. Additional active agents that could be delivered by means of the composition of the invention are set out in the following non-limiting list:
Antimalarial agents (e.g. artemisinin derivatives, dihydroartemisinin, artemotil, chloroquine, primaquine, doxycillin, quinine, aminoquinolines, cinchona alkaloids, antifolates, quinidine, mefloquine, halofantrine, lumefantrine, amodiaquine, pyronaridine, tafenoquine, artesunate, artemether, biguanides, proguanil, chloproguanil, diaminopyrimidines, pyrimethamine, trimethoprim, dapsone, sulfonamides, atovaquone, sulfadoxine-pyrimethamine, N-acetyl cysteine, piperaquine, DHA-piperaquine, dermaseptins, bisphosphonates, quercetin etc. The drugs could be used alone or in combinations.)
OTC drugs (e.g. antipyretics, anesthetics, cough suppressants, etc.)
Anti-infective agents
Anti-malaria agents (such as dihydroartemisinin, etc.)
Antibiotics (e.g. penicillins, cephalosporins, macrolides, tetracyclines, aminoglycosides, anti-tuberculosis agents, doxycycline, ciprofloxacin, moxifloxacin, gatifloxacine, carbapenems, azithromycin, clarithromycin, erythromycin, ketolides, penems, tobramycin, filgrastim, pentamidine, microcidin, clerocidin, amikacine, etc.)
Genetic molecules (e.g. Anti-sense oligonucleotides, nucleic acids, oligonucleotides, DNA, RNA,
Anti-cancer agents (e.g. anti-proliferative agents, anti-vascularization agents, taxol, etopside, cisplatin, etc.)
Anti-protozoal agents
Antivirals (e.g. acyclovir, ganciclovir, ribavirin, anti-HIV agents, anti-hepatitis agents, famciclovir, valaciclovir, didanosine, saquinavir, ritonavir, lamivudine, stavudine, zidovudine, etc.)
Anti-inflammatory drugs (e.g. NSAIDs, steroidal agents, cannabinoids, leukotriene-antagonists, tacrolimus, sirolimus, everolimus, etc.)
Anti-allergic molecules (e.g. antihistamines, fexofenadine)
Bronchodilators
Vaccines and other immunogenic molecules (e.g. tetanus toxoid, reduced diphtheria toxoid, acellular pertussis vaccine, mumps vaccine, smallpox vaccine, anti-HIV vaccines, hepatitis vaccines, pneumonia vaccines, influenza vaccines, TNF-alpha-antibodies etc.)
Anesthetics, local anesthetics.
Antipyretics (e.g. paracetamol, ibuprofen, diclofenac, aspirin, etc.)
Agents for treatment of severe events such cardiovascular attacks, seizures, hypoglycemia, etc.
Afrodisiacs from plants or synthetics
Anti-nausea and anti-vomiting.
Immunomodulators (immunoglobulins, etc.)
Cardiovascular drugs (e.g. beta-blockers, alpha-blockers, calcium channel blockers, etc.)
steroid hormones (eg. insulin, insulin derivatives, insulin detemir, insulin monomeric, oxytocin, LHRH, LHRH analogues, adreno-corticotropic hormone, somatropin, leuprolide, calcitonin, parathyroid hormone, estrogens, testosterone, adrenal corticosteroids, megestrol, progesterone, sex hormones, growth hormones, growth factors, etc.)
Vitamins (e.g. Vit A, Vitamins from B group, folic acid, Vit C, Vit D, Vit E, Vit K, niacin, derivatives of Vit D, etc.)
Autonomic Nervous System Drugs
Fertilizing agents
Antidepressants (e.g. buspirone, venlafaxine, benzodiazepines, selective serotonin reuptake inhibitors (SSRIs), sertraline, citalopram, tricyclic antidepressants, paroxetine, trazodone, lithium, bupropion, sertraline, fluoxetine, etc.)
Agents for smoking cessation (e.g. bupropion, nicotine, etc.)
Agents for treating alcoholism and alcohol withdrawal
Lipid-lowering agents (eg. inhibitors of 3 hydroxy-3-methylglutaryl-coenzyme A (HMG-CoA) reductase, simvastatin, atorvastatin, etc.)
Drugs for CNS or spinal cord (benzodiazepines, lorazepam, hydromorphone, midazolam, Acetaminophen, 4′-hydroxyacetanilide, barbiturates, anesthetics, etc.)
Anti-epilepsic agents (e.g. valproic acid and its derivatives, carbamazepine, etc.)
Angiotensin antagonists (e.g. valsartan, etc.)
Anti-psychotic agents and anti-schizophrenic agents (e.g. quetiapine, risperidone)
Agents for treatment of Parkinsonian syndrome (e.g. L-dopa and its derivatives, trihexyphenidyl, etc.)
Anti-Alzheimer drugs (e.g. cholinesterase inhibitors, galantamine, rivastigmine, donepezil, tacrine, memantine, N-methyl D-aspartate (NMDA) antagonists).
Agents for treatment of non-insulin dependent diabetes (e.g. metformin,
Agents against erectile dysfunction (e.g. sildenafil, tadalafil, papaverine, vardenafil, PGE1, etc.)
Prostaglandins
Agents for bladder dysfunction (e.g. oxybutynin, propantheline bromide, trospium, solifenacin succinate etc.)
Agents for treatment menopausal syndrome (e.g estrogens, non-estrogen compounds, etc.)
Agents for treatment hot flashes in postmenopausal women
Agents for treatment primary or secondary hypogonadism (e.g. testosterone, etc.)
Cytokines (e.g. TNF, interferons, IFN-alpha, IFN-beta, interleukins etc.)
CNS stimulants
Muscle relaxants
Anti paralytic gas agents
Appetite stimulators/depressors (e.g. cannabinoids, etc.)
Narcotics and Antagonists (e.g. opiates, oxycodone etc.)
Painkillers (opiates, endorphins, tramadol, codeine, NSAIDs, gabapentin, fentanyl and pharmaceutically acceptable salts thereof etc.)
Hypnotics (Zolpidem, benzodiazepins, barbiturates, ramelteon, etc.)
Histamines and Antihistamines
Antimigraine Drugs (e.g. imipramine, propranolol, sumatriptan, eg.)
Diagnostic agents (e.g. Phenolsulfonphthalein, Dye T-1824, Vital Dyes, Potassium Ferrocyanide, Secretin, Pentagastrin, Cerulein, etc.)
Topical decongestants or anti-inflammatory drugs
Anti-acne agents (e.g. retinoic acid derivatives, doxycycline, minocycline, etc.)
ADHD related medication (e.g. methylphenidate, dexmethylphenidate, dextroamphetamine, d- and 1-amphetamine racemic mixture, pemoline, etc.)
Diuretic agents
Anti-osteoporotic agents (e.g. bisphosphonates, alendronate, pamidronate, tirphostins, etc.)
Drugs for treatment of asthma drugs for post trauma, crisis, anxiety treatment
Anti-spasmodic agents (e.g. papaverine, etc.)
Agents for treatment of multiple sclerosis and other neurodegenerative disorders (e.g. mitoxantrone, glatiramer acetate, interferon beta-la, interferon beta-1b, etc.)
Plant derived agents from leave, root, flower, seed, stem or branches extracts.
Vitamins and food supplements
Veterinary use; the compositions of the invention can be given alone or in combination with another active ingredient to animals including cattle and pets for management of several medical conditions. For example, they can be used to reduce anxiety, stress and inflammation, to relieve pain, to stimulate appetite, to control neurologic conditions and to improve reproductive efficiency.

Due to the efficiency of the compositions of the invention, the concentration and amount (e.g., dosage unit) of the formulation may be readily adjusted such that its delivery comply with the selected dosage regimen. A therapeutically effective amount the active ingredient in the methods of treatment provided by the present invention may be from 10 mcg to 1000 mg per kg body weight of the patient treated by the methods described above, per day, e.g., from 10, 25, 50, 75, 100, 150, 200, 300 mcg per kg per day up to 1, 10 100, 500, 600, 700, 800, 900 and 1000 mg/kg/day.

The compositions of the invention can be administered not only in capsules, e.g., as oral solution or oral drops. Furthermore, although useful as pharmaceutical compositions, the composition of the invention may also be used as nutritional composition, food supplement, pets and cattle administrable products.

### IN THE DRAWINGS

FIG. 1. Mean writing counts in mice treated with 50 mg/kg CBD per os from the new oral liquid composition as compared to control oral composition each containing 32% w/w, 0.5, 2, 4 and 6 hours prior to IP injection of acetic acid and compared to untreated control mice received IP injection of acetic acid, n=4/group. (Mean±SD). p<0.05 and <0.01 for new oral liquid composition vs. control oral composition at 0.5 and 2 hour time points respectively. p<0.001 for new oral liquid composition vs untreated control at 0.5, 2 and 6 time points, p<0.01 for new oral liquid composition vs untreated control at 4 hours time point. p<0.01 for control oral composition vs untreated control at 0.5, 4 and 6 time points, p<0.05 for control oral composition vs untreated control at 2 hours time point, by one-way ANOVA

FIG. 2. MPE % values in mice treated with 50 mg/kg CBD per os from the new oral liquid composition as compared to control oral composition each containing 32% w/w, 0.5, 2, 4 and 6 hours prior to IP injection of acetic acid

FIG. 3. Representative graph for the behavior of the capsules filled with two CBD compositions (liquid and solid) after incubation at 37° C. with shaking in simulated gastric fluid for 2 h, and followed by incubation in intestinal fluid for 22 h.

### EXAMPLES

Glossary: PL—phospholipids; PG—propylene glycol; CBD—Cannabidiol; THC—Tetrahydrocannabinol; CBN—Cannabinol; HSO—hemp seed oil; Vit E—vitamin E; Fluorescein isothiocyanate: FITC. CBD obtained by extraction from plants or synthetic. THC obtained by extraction from plant, Lipoid S100 and Phospholipon® 90 G are from Lipoid GmbH, Germany. Sunlipon® 90 is from Perimondo, USA. Pomegranate Oil (organic) manufactured by Bara Herbs, Israel and Hemp Seed Oil (organic) manufactured by Pukka Herbs, UK were used. Lecithin Soya (Fagron, Spain) and Propylene glycol (Tamar) from Tamar, Israel. Olive Oil from Henry Lamotte Oil GmbH, Germany.

### Example 1

TABLE 1

| Ingredients | Concentration % w/w |
|---|---|
| CBD | 41.3 |
| Phospholipon ® 90G | 41.3 |
| Vitamin E | 1.3 |
| Propylene glycol | 16.1 |

Preparation: PL was mixed well, then CBD was added and mixed well. Vitamin E was added and mixed. Finally, PG was added with mixing. A viscous liquid was obtained.

### Example 2

TABLE 2

| Ingredients | Concentration % w/w |
|---|---|
| CBD | 39.3 |
| Phospholipon ® 90G | 46.8 |
| Vitamin E | 1.4 |
| Hemp seed oil | 4.7 |
| Propylene glycol | 7.8 |

Preparation: PL was mixed well then CBD was added and mixed well. Then Vit E was added, followed by the addition of HSO with mixing. Finally, PG was added and mixed. A viscous liquid was obtained.

### Example 3

TABLE 3

| Ingredients | Concentration % w/w |
|---|---|
| CBD | 42.1 |
| Phospholipon ® 90G | 42.1 |
| Vitamin E | 1.5 |
| Hemp seed oil | 4.3 |
| menthol | 0.2 |
| Propylene glycol | 9.8 |

Preparation: PL was mixed well then CBD was added and mixed well. Then Vitamin E was added followed by addition of HSO with mixing. Then menthol was added and mixed well. Finally, PG was added with mixing. A viscous liquid was obtained.

### Example 4

TABLE 4

| Ingredients | Concentration % w/w |
|---|---|
| CBD | 37.8 |
| Phospholipon ® 90H | 37.8 |
| Vitamin E | 1.3 |
| Olive oil | 9.5 |
| Propylene glycol | 13.6 |

Preparation: Phospholipon® 90H was mixed well then CBD was added and mixed well. Then Vitamin E was added, followed by olive oil addition with mixing. Finally, PG was added with mixing. A viscous liquid was obtained.

### Example 5

TABLE 5

| Ingredients | Concentration % w/w |
|---|---|
| CBD | 32 |
| Phospholipon ® 90G | 32 |
| Vitamin E | 0.5 |
| Propylene glycol | 35.5 |

Preparation: PL was mixed well then CBD was added and mixed well. Then Vit E is added and mixed. Finally, PG is added and mixed. A liquid was obtained.

### Example 6

TABLE 6

| Ingredients | Concentration % w/w |
|---|---|
| CBD | 40 |
| Phospholipon ® 90G | 40 |
| Vitamin E | 0.8 |
| Propylene glycol | 19.2 |

Preparation: PL was mixed well then CBD was added and mixed well. Then Vit E is added with mixing. Finally, PG is added and mixed. A viscous liquid was obtained.

### Example 7

TABLE 7

| Ingredients | Concentration % w/w |
|---|---|
| CBD | 45 |
| Phospholipon ® 90G | 37.8 |
| Vitamin E | 1.3 |
| Olive oil | 4 |
| Propylene glycol | 11.9 |

Preparation: PL was mixed well then CBD was added and mixed well. Then Vit E was added followed by olive oil addition with mixing. Finally, PG was added and mixed.

### Example 8

TABLE 8

| Ingredients | Concentration % w/w |
|---|---|
| CBD | 40 |
| Lipoid S | 40 |
| Vitamin E | 0.8 |
| Propylene glycol | 19.2 |

Preparation: Lipoid was mixed well, then CBD was added and mixed well. Then Vitamin E was added and mixed. Finally, PG was added and mixed. A viscous liquid was obtained.

### Example 9

TABLE 9

| Ingredients | Concentration % w/w |
|---|---|
| CBD | 30 |
| THC | 10 |
| Lipoid S | 40 |
| Vitamin E | 0.8 |
| Propylene glycol | 19.2 |

Preparation: Lipoid was mixed well with CBD and THC. Then Vitamin E was added with mixing. Finally, PG was added and mixed.

### Example 10

TABLE 10

| Ingredients | Concentration % w/w |
|---|---|
| CBD | 35 |
| THC | 5 |
| Lipoid S | 40 |
| Vitamin E | 0.8 |
| Propylene glycol | 19.2 |

Preparation: Lipoid was mixed well with CBD and THC. Then Vitamin E was added with mixing. Finally, PG was added and mixed. A viscous liquid was obtained.

### Example 11

TABLE 11

| Ingredients | Concentration % w/w |
|---|---|
| CBD | 39 |
| THC | 1 |
| Lipoid S | 40 |
| Vitamin E | 1 |
| Propylene glycol | 19 |

Preparation: Lipoid was mixed well with CBD and THC. Vitamin E was added with mixing. Finally, PG was added and mixed.

### Example 12

TABLE 12

| Ingredients | Concentration % w/w |
|---|---|
| CBD | 30 |
| THC | 20 |
| Phospholipon G | 40 |
| Vitamin E | 0.8 |
| Propylene glycol | 9.2 |

Preparation: Phospholipon G was mixed well with CBD and THC. Vitamin E was added with mixing. Finally, PG was added and mixed. A liquid was obtained.

### Example 13

TABLE 13

| Ingredients | Concentration % w/w |
|---|---|
| CBD | 30 |
| THC | 10 |
| Lipoid S | 10 |
| Phospholipon ® G | 20 |
| Vitamin E | 0.8 |
| Propylene glycol | 29.2 |

Preparation: Lipoid and Phospholipon® G are mixed well with CBD and THC. Then Vitamin E was added with mixing. Finally, PG was added and mixed. A liquid was obtained.

### Example 14

TABLE 14

| Ingredients | Concentration % w/w |
|---|---|
| CBD | 30 |
| THC | 5 |
| CBN | 10 |
| Phospholipon G | 40 |
| Vitamin E | 0.8 |
| Propylene glycol | 14.2 |

Preparation: Phospholipon® G is mixed well with the mixture of drugs (CBD, THC and CBN). Then Vitamin E was added with mixing. Finally, PG was added and mixed.

### Examples 15 to 19

TABLE 15

| | Exam- | Exam- | Exam- | Exam- | Exam- |
|---|---|---|---|---|---|
| | ple 15 | ple 16 | ple 17 | ple 18 | ple 19 |
| Ingredient | wt % | wt % | wt % | wt % | wt % |
| CBD | 40 | 30 | 30 | 30 | 30 |
| Tramadol HCl | 1 | — | — | — | — |
| Diazepam | — | 1 | 1 | — | — |
| Brotizolam | — | — | — | 0.25 | — |
| Butarphenol | | | | | 0.5 |
| Phospholipon ® | 35 | 25 | — | 30 | 30 |
| 90G | | | | | |
| Lipoid PC | — | 7 | 15 | — | 5 |
| Phospholipon ® | — | — | 15 | — | — |
| 90H | | | | | |
| Vitamin E | 1 | q.s. | q.s. | 0.75 | 0.5 |
| Propylene | 23 | to 100 | to 100 | 39 | 34 |
| glycol | | | | | |

The compositions of Examples 15 to 19 set out in Table 15 were prepared using the procedures described in previous examples. Phospholipid(s) are combined with the cannabinoids and mixed well, followed by addition of the other drug with mixing, and addition of the antioxidant and PG under mixing.

### Examples 20-22

This set of examples illustrate the incorporation of pomegranate oil into formulations of the invention; the total concentrations of the cannabinoid(s) and the oil in the illustrated formulations is from ˜36% to 45% by weight.

TABLE 16

| | 20 | 21 | 22 |
|---|---|---|---|
| Ingredient | wt % | | |
| Lipoid S100 | 50.0 | 40.0 | |
| Phospholipon ® 90G | | | 35.0 |
| CBD | 25.0 | 29.0 | 30.0 |
| CBN | | 1.0 | |
| Pomegranate oil | 12.5 | 15.0 | 6.0 |
| Propylene glycol | 12.5 | 15.0 | 27.0 |

The compositions set out in Table 16 were prepared by the procedures described above. In Examples 20 and 21, propylene glycol was the last added ingredient: it was slowly added under stirring to the phospholipids, cannabinoid(s) and oil mixture. In Example 22, the order of addition was different: Phospholipon® 90G was mixed with the CBD and CBN, followed by the addition of propylene glycol; the pomegranate oil was then added slowly under mixing. In all three cases, homogenous (brownish or yellowish) viscous liquid is obtained.

### Examples 23-28

This set of Examples illustrate the incorporation of cannabinoid (either a single cannabinoid or a mixture of two cannabinoids) and therapeutically effective oils into high content phospholipids preparations.

TABLE 17

| | 23 | 24 | 25 | 26 | 27 | 28 |
|---|---|---|---|---|---|---|
| | Wt % | Wt % | Wt % | Wt % | Wt % | Wt % |
| Lipoid S100 | | 50.0 | | | | 40.0 |
| Lecithin soya | 33.3 | | 40 | 44 | 40 | |
| CBD | 29.0 | 29.0 | 20 | 30 | 30 | 30 |
| THC | 1.0 | 1.5 | 10 | | | |
| Pomegranate oil | 36.7 | 18.5 | 15 | 10 | 15 | 15 |
| Brassica oil | | | | | | |
| Black sesame | | | | | | 10 |
| seed oil | | | | | | |
| Olive oil | | | 15 | 16 | | |
| Hemp seed oil | | | | | 15 | 5 |

The compositions are prepared by first mixing the phospholipids component. Then the cannabinoid(s) are added with mixing, followed by slow or portion wise addition of the oil(s) under mixing. When a mixture of oils is used, the oils are added either successively (e.g., a portion of one oil is mixed with the phospholipids/cannabinoids, followed by slow addition under mixing of the remaining portion and the other oil or by successive addition of the oils to the phospholipids/cannabinoids with mixing. Homogeneous brownish liquids or viscous liquids are formed.

### Example 29

TABLE 18

| Ingredients | Concentration % w/w |
|---|---|
| CBD | 67 |
| Sunlipon ® 90 | 32.5 |
| Vitamin E | 0.5 |

Preparation: Sunlipon was mixed well, then CBD was added and mixed well. A viscous liquid was obtained. Then Vitamin E was added and mixed. A viscous liquid was obtained.

### Example 30

TABLE 19

| Ingredients | Concentration % w/w |
|---|---|
| CBD | 67 |
| Phospholipon ® 90G | 32.2 |
| Vitamin E | 0.8 |

Preparation: Phospholipon® 90G was mixed well, then CBD was added and mixed well. A viscous liquid was obtained. Then Vitamin E was added and mixed.

### Example 31

TABLE 20

| Ingredients | Concentration % w/w |
|---|---|
| CBD | 60 |
| Phospholipon ® 90G | 39.2 |
| Vitamin E | 0.8 |

Preparation: Phospholipon® 90G was mixed well, then CBD was added and mixed well. A viscous liquid was obtained. Then Vitamin E was added and mixed. A viscous liquid was obtained.

### Example 32

TABLE 21

| Ingredients | Concentration % w/w |
|---|---|
| CBD | 75 |
| Phospholipon ® 90G | 24 |
| Vitamin E | 1 |

Preparation: Phospholipon® 90G was mixed well, then CBD was added and mixed well. A viscous liquid was obtained. Then Vitamin E was added and mixed. A viscous liquid was obtained.

### Example 33

TABLE 22

| Ingredients | Concentration % w/w |
|---|---|
| CBD | 79 |
| Sunlipon ® 90 | 20 |
| Vitamin E | 1 |

Preparation: Sunlipon® 90 was mixed well, then CBD was added and mixed well. A viscous liquid was obtained. Then Vitamin E was added and mixed. A viscous liquid was obtained.

### Example 34

TABLE 23

| Ingredients | Concentration % w/w |
|---|---|
| CBD | 57 |
| Phospholipon ® 90G | 37 |
| Vitamin E | 0.8 |
| Propylene glycol | 5.2 |

Preparation: Phospholipon® 90G is mixed well, then CBD is added and mixed well. A viscous liquid is obtained. Then Vitamin E is added and mixed. Finally, PG is added and mixed. A viscous liquid is obtained.

### Example 35

TABLE 24

| Ingredients | Concentration % w/w |
|---|---|
| CBD | 44 |
| Lipoid S100 | 55 |
| Vitamin E | 1 |

Preparation: Lipoid is mixed well, then CBD is added and mixed well. A viscous liquid is obtained. Then Vitamin E is added and mixed. A viscous liquid is obtained.

### Example 36

TABLE 25

| Ingredients | Concentration % w/w |
|---|---|
| CBD | 42 |
| Sunlipon ® 90 | 57 |
| Vitamin E | 1 |

Preparation: Sunlipon® 90 is mixed well, then CBD is added and mixed well. A viscous liquid is obtained. Then Vitamin E is added and mixed. A viscous liquid is obtained.

### Example 37

TABLE 26

| Ingredients | Concentration % w/w |
|---|---|
| CBD | 59.5 |
| Lipoid S | 39.5 |
| Vitamin E | 1.0 |

Preparation: Lipoid is mixed well, then CBD is added and mixed well. A liquid is obtained. Then Vitamin E is added and mixed. A viscous liquid is obtained.

### Example 38

TABLE 27

| Ingredients | Concentration % w/w |
|---|---|
| CBD | 41.3 |
| Phospholipon ® 90G | 41.3 |
| Vitamin E | 1.3 |
| Propylene glycol | 16.1 |

Preparation: PL was mixed well, then CBD was added and mixed well. A viscous liquid was obtained. Vitamin E was added and mixed. Finally, PG was added with mixing. A liquid was obtained.

### Example 39

TABLE 28

| Ingredients | Concentration % w/w |
|---|---|
| CBD | 39.3 |
| Phospholipon ® 90G | 46.8 |
| Vitamin E | 1.4 |
| Hemp seed oil | 4.7 |
| Propylene glycol | 7.8 |

Preparation: PL was mixed well then CBD was added and mixed well. A viscous liquid was obtained. Then Vit E was added, followed by the addition of HSO with mixing. Finally, PG was added and mixed. A viscous liquid was obtained.

### Example 40

TABLE 29

| Ingredients | Concentration % w/w |
|---|---|
| CBD | 44 |
| Phospholipon ® 90G | 40.2 |
| Vitamin E | 1.5 |
| Hemp seed oil | 4.3 |
| Menthol | 0.2 |
| Propylene glycol | 9.8 |

Preparation: PL was mixed well then CBD was added and mixed well. A viscous liquid was obtained. Then Vitamin E was added followed by addition of HSO with mixing. Then menthol was added and mixed well. Finally, PG was added with mixing. A viscous liquid was obtained.

### Example 41

TABLE 30

| Ingredients | Concentration % w/w |
|---|---|
| CBD | 39.0 |
| Phospholipon ® 90H | 36.6 |
| Vitamin E | 1.3 |
| Olive oil | 9.5 |
| Propylene glycol | 13.6 |

Preparation: Phospholipon® 90H was mixed well then CBD was added and mixed well. A viscous liquid was obtained. Then Vitamin E was added, followed by olive oil addition with mixing. Finally, PG was added with mixing. A viscous liquid was obtained.

### Example 42

TABLE 31

| Ingredients | Concentration % w/w |
|---|---|
| CBD | 32.9 |
| Phospholipon ® 90G | 31.1 |
| Vitamin E | 0.5 |
| Propylene glycol | 35.5 |

Preparation: PL was mixed well, then CBD was added and mixed well. A viscous liquid was obtained. Then Vit E was added and mixed. Finally, PG was added and mixed. A liquid was obtained.

### Example 43

TABLE 32

| Ingredients | Concentration % w/w |
|---|---|
| CBD | 49.0 |
| Phospholipon ® 90G | 40.0 |
| Vitamin E | 0.8 |
| Propylene glycol | 10.2 |

Preparation: PL was mixed well then CBD was added and mixed well. A viscous liquid was obtained. Then Vit E was added with mixing. Finally, PG was added and mixed. A viscous liquid was obtained.

### Example 44

TABLE 33

| Ingredients | Concentration % w/w |
|---|---|
| CBD | 45 |
| Phospholipon ® 90G | 37.8 |
| Vitamin E | 1.3 |
| Olive oil | 4 |
| Propylene glycol | 11.9 |

Preparation: PL was mixed well then CBD was added and mixed well. A liquid was obtained. Then Vit E was added followed by olive oil addition with mixing. Finally, PG was added and mixed.

### Example 45

TABLE 34

| Ingredients | Concentration % w/w |
|---|---|
| CBD | 50 |
| Lipoid S | 35 |
| Vitamin E | 0.8 |
| Propylene glycol | 14.2 |

Preparation: Lipoid was worked well, then CBD was added and mixed well. A liquid was obtained. Then Vitamin E was added and mixed. Finally, PG was added and mixed. A viscous liquid was obtained.

### Example 46

TABLE 35

| Ingredients | Concentration % w/w |
|---|---|
| CBD | 32 |
| THC | 10 |
| Lipoid S | 38 |
| Vitamin E | 0.8 |
| Propylene glycol | 19.2 |

Preparation: Lipoid was mixed well. Then CBD and THC were added. A liquid was obtained with mixing. Then Vitamin E was added with mixing. Finally, PG was added and mixed.

### Example 47

TABLE 36

| Ingredients | Concentration % w/w |
|---|---|
| CBD | 35 |
| THC | 7 |
| Lipoid S | 57.2 |
| Vitamin E | 0.8 |

Preparation: Lipoid was mixed well, then CBD and THC were added with mixing. A liquid was obtained. Then Vitamin E was added with mixing. A viscous liquid was obtained.

### Example 48

TABLE 37

| Ingredients | Concentration % w/w |
|---|---|
| CBD | 36 |
| THC | 10 |
| Sunlipon ® 90 | 34 |
| Vitamin E | 0.8 |
| Propylene glycol | 19.2 |

Preparation: Lipoid is mixed well then CBD and THC are added with mixing and continuing mixing till a viscous liquid is obtained. Then Vitamin E is added with mixing. Finally, PG is added and mixed. A viscous liquid is obtained.

### Example 49

TABLE 38

| Ingredients | Concentration % w/w |
|---|---|
| CBD | 39 |
| THC | 4 |
| Lipoid S | 37 |
| Vitamin E | 1 |
| Propylene glycol | 19 |

Preparation: Lipoid was mixed well with CBD and THC. Vitamin E was added with mixing. Finally, PG was added and mixed. A viscous liquid was obtained.

### Example 50

TABLE 39

| Ingredients | Concentration % w/w |
|---|---|
| CBD | 30 |
| THC | 10 |
| Lipoid S | 10 |
| Phospholipon ® 90G | 20 |
| Vitamin E | 0.8 |
| Propylene glycol | 29.2 |

Preparation: Lipoid and Phospholipon® 90G are mixed well. Then CBD and THC are added with thoroughly mixing. A liquid is obtained. Then Vitamin E is added with mixing. Finally, PG is added and mixed. A liquid is obtained.

### Example 51

TABLE 40

| Ingredients | Concentration % w/w |
|---|---|
| CBD | 30 |
| THC | 14.2 |
| CBN | 10 |
| Phospholipon ® 90G | 40 |
| Vitamin E | 0.8 |
| Propylene glycol | 5.0 |

Preparation: Phospholipon® 90G is mixed well. Then CBD, THC and CBN are added through mixing. The mixture is further mixed. A viscous liquid is obtained. Then Vitamin E is added with mixing. Finally, PG is added and mixed.

### Examples 52 to 57

TABLE 41
Compositions of CBD with another active compound

| | Composition | | | | | |
|---|---|---|---|---|---|---|
| | Example | Example | Example | Example | Example | Example |
| | 52 | 53 | 54 | 55 | 56 | 57 |
| Ingredient | wt % | wt % | wt % | wt % | wt % | wt % |
| CBD | 40 | 30 | 30 | 30 | 30 | 30 |
| Paracetamol | 5 | — | — | — | — | — |
| Ibuprofen | — | 5 | — | — | — | — |
| Zolmitriptan | — | — | 1 | — | — | — |
| Rizatriptan Benzoate | — | — | — | 1 | — | — |
| Ondansetron | — | — | — | — | 1 | — |
| Granisetron | — | | | | | 1 |
| Phospholipon ® 90G | 30 | 25 | — | 28 | — | — |
| Lipoid PC | — | 7 | 15 | — | — | — |
| Sunlipon ® 90 | — | — | 20 | — | 35 | 30 |
| Vitamin E | 0.8 | 1 | 0.75 | q.s. | 1 | 1 |
| Propylene glycol | to 100 | to 100 | to 100 | to 100 | to 100 | to 100 |

The compositions of Examples 52 to 57 set out in the above Table are prepared using the procedures described in previous examples. Phospholipid(s) are mixed well, the cannabinoids are added and mixed well, followed by addition of the other active ingredient with mixing, and addition of the antioxidant and PG under mixing.

### Examples 58-60

This set of examples illustrate the incorporation of oil into formulations of the invention.

TABLE 42

| | Composition | | |
|---|---|---|---|
| | Example 58 | Example 59 | Example 60 |
| Ingredient | wt % | | |
| Lipoid S100 | 55.0 | — | — |
| Phospholipon ® 90G | — | — | 45.0 |
| Sunlipon ® 90 | — | 40.0 | — |
| CBD | 38.0 | 49.0 | 40.0 |
| CBN | — | 1.0 | — |
| Pomegranate oil | 7 | 5.0 | 6.0 |
| Propylene glycol | — | 5.0 | 9.0 |

The compositions set out in the above Table were prepared by the procedures described above.

### Example 61

### In Vivo Analgesic Effect of CBD Incorporated in the Oral Liquid Composition of the Invention, Measured in Pain Animal Model

In this experiment the antinociceptive effect of CBD administered in the novel oral liquid composition was measured and compared to a control formulation.

### Compositions

### Oral Liquid Composition of the Invention (Example 14)

TABLE 43

| Ingredient | % w/w |
|---|---|
| CBD | 32.9 |
| Phospholipon ® 90G | 31.1 |
| Vit E | 0.5 |
| PG | 35.5 |

Preparation: Phospholipon® 90G was mixed well, then CBD was added with mixing. A viscous liquid was obtained. Then Vitamin E was added with mixing. Finally, PG was added and the composition was mixed.

### Control Oral Composition

TABLE 44

| Ingredient | % w/w |
|---|---|
| CBD | 32 |
| Olive oil | 68 |

Preparation: CBD was mixed well with olive oil.

### Experimental Protocol

### Animals

This experiment was performed on 36 male CD-1 ICR mice (27-30 g). Mice were housed under standard conditions of light and temperature in plastic cages in the specific-pathogen unit (SPF) of the pharmacy school at the Hebrew University of Jerusalem. Animals were kept in separated cages with smooth flat floor and provided with unlimited access to water and food.

### Treatments and Writhing Test

The mice were divided randomly to eight treatment groups, each consisting of four animals, to test the two compositions at four time points (0.5, 2, 4 and 6 hours). The animals were treated orally with ˜4.5 mg (˜5 μl) of the new oral liquid composition and the control composition at CBD dose of 50 mg/kg. The oral administration of the compositions was performed using a positive displacement pipette and CP25 capillaries and pistons (MICROMAN®, precision microliter pipette, GILSON, France). The filled capillary was gently inserted into the oral cavity until it reached the pharynx, then the composition was slowly released. At the predetermined time points (0.5, 2, 4 and 6 hours following the treatments), the animals were injected intraperitoneally with acetic acid (0.6% v/v) at a dose of (10 ml/kg) (n=4).

The four remaining animals anesthetized with Isoflurane® were injected with acetic acid at the same dose without treatment served as untreated control.

Number of writhing episodes was recorded by counting the number of writhes 5 minutes after acetic acid administration for a period of 20 minutes. Writhes are indicated by the abdominal constriction and stretching of at least one hind limb.

The analgesic effect of each treatment is expressed by the Maximum Possible Effect (MPE %), which is directly related to the efficiency of the treatment, and is calculated according to the following equation:

MPE %=[Mean of writhes count in untreated control group−mean writhes count in treated group]/[Mean of writhes in untreated control group]*100

### Results

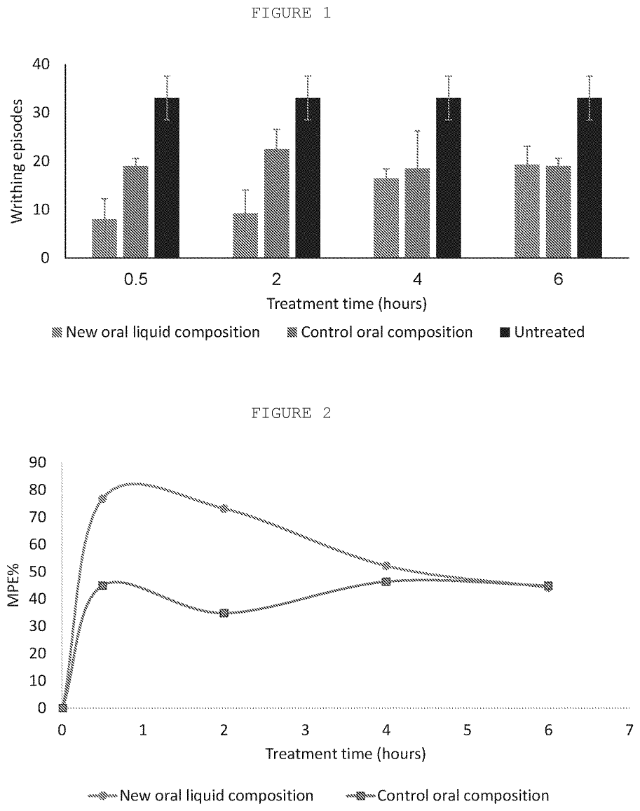

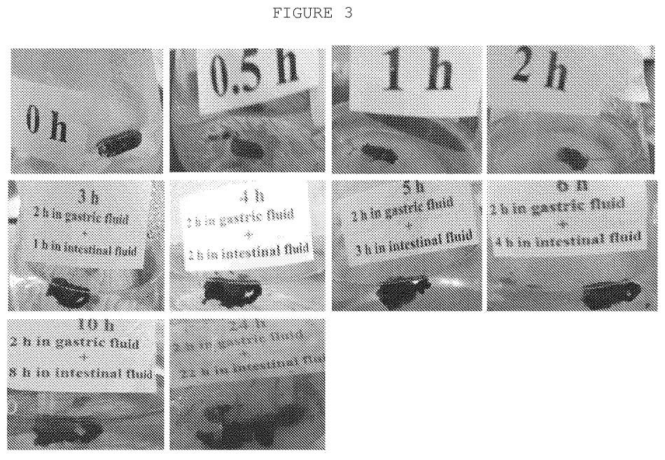

Results are presented in Tables A and B and FIGS. 1 and 2 respectively.

Mean writing counts in mice treated with 50 mg/kg CBD per as from the new oral liquid composition as compared to control oral composition (each containing 32% w/w CBD), at 0.5, 2, 4 and 6 hours prior to IP injection of acetic acid are set out in Table 1 and compared to untreated control mice which received IP injection of acetic acid, n=4/group, (Mean±SD).

TABLE 45

| Time point | Writhes Count | | | |
|---|---|---|---|---|
| (hours) | 0.5 | 2 | 4 | 6 |
| New oral liquid | 8.0 ± 4.2 | 9.25 ± 4.8 | 16.5 ± 1.9 | 19.3 ± 3.8 |
| composition | | | | |
| Control oral | 19.0 ± 1.5 | 22.5 ± 4.04 | 18.5 ± 7.8 | 19 ± 1.5 |
| composition | | | | |

| | |
|---|---|
| Untreated | 33.0 ± 4.5 |
| control | |

The calculated MPE % values are tabulated in Table B.

TABLE 46

MPE %

| | 0.5 | 2 | 4 | 6 |
|---|---|---|---|---|
| New oral liquid | 76.8 | 73.2 | 52.1 | 44.2 |
| composition | | | | |
| Control oral | 44.9 | 34.8 | 46.4 | 44.9 |
| composition | | | | |

### Conclusion

CBD administration to mice from the oral liquid composition of the invention has led to decrease of the writhing counts from 33.0±4.5 in the untreated mice group to less than 10 writhes during the first two hours following the treatment. These excellent results indicate a rapid and significant analgesic effect of the new system showing a very high 76.8 and 73.2% MPE, after 0.5 and 2 hours, respectively. At these time points, the calculated MPE values for the animals that received the same CBD dose by the control oral composition were only 44.9 and 34.8%, respectively.

### Example 62

### Disintegration Test on Capsules Incorporating the Liquid CBD Composition of the Invention and the Solid Composition of WO 2017/098502.

The study reported in this example shows the disintegration behavior of a dosage form based on hard gelatin capsule filled with:

CBD liquid composition of the present invention, to which Methylene Blue dye was added, and
CBD solid composition described in WO 2017/098502, to which Sudan III dye was added.

The dosage form was incubated in simulated gastric fluid for 2 h followed by incubation in simulated intestinal fluid for additional 22 h with shaking. Photographs were taken over the twenty four hours test period to examine the behavior of the dosage form.

### Compositions

### Oral Liquid Composition of the Invention

TABLE 47

| Ingredient | % w/w |
|---|---|
| CBD | 42 |
| Phospholipon ® 90G | 38 |
| Vit E | 1 |
| PG | 18.5 |
| Methylene blue | 0.5 |

Preparation: Phospholipon® 90G was mixed well, then CBD was added and mixed well. A viscous liquid was obtained. Then Vitamin E was added and mixed. Finally, PG was added and mixed. Methylene blue was then added and mixed well. A viscous dark blue liquid was obtained.

### Oral Solid Composition of WO 2017/098502.

TABLE 48

| Ingredient | % w/w |
|---|---|
| CBD | 10 |
| Phospholipon ® 90G | 89 |
| Vit E | 0.5 |
| Sudan III | 0.5 |

Preparation: Phospholipon® 90G was worked well then mixed with CBD. Vit E was added and mixed well. Sudan III was then added and mixed well. A solid red mass was obtained

### Final Dosage Form Capsules

Hard gelatin capsules were used in this experiment. Part of the space of the hard capsule was occupied by the liquid composition. The solid composition of WO 2017/098502 was placed in the hard capsule. The capsules were then closed tightly.

### Experimental Protocol

The instrument used was Gyratory water bath shaker, model G76 (New Brunswick Scientific Co, INC, USA). Test conditions:

Bath temperature: 37′C.
Shaker speed: 1
Incubation medium: simulated gastric fluid (0.5% v/v HCl solution, pH 1-2), simulated intestinal fluid (0.68% w/v KH2PO4, 0.089% w/v NaOH, pH 6-7)

The capsules were incubated in flasks containing 200 ml simulated gastric fluid for the first 2 h, then transferred to other flasks containing 200 ml simulated intestinal fluid for additional 22 h.

### Results

The photographs taken during the twenty four hours test period (incubation in gastric medium for two hours, followed by additional twenty-two hours in intestinal fluid under shaking) are presented in FIG. 3. Photographs were taken at t=0, 0.5 h, 1 h, 2 h (top row, left to right), t=3, 4 h, 5 h, 6 h (second row, left to right) and t=10 h and 24 h (bottom row, left to right).

The gelatin shells of the capsules dissolved during the first half hour of the experiment. During this time, the capsules' wall containing the oral liquid composition (dyed with methylene blue) started to disintegrate. This led to blue coloring of the incubation medium, owing to release of the liquid composition of the invention. In contrast, the solid composition of composition dyed with Sudan III was eroded but non-disintegrated till the end of the experiment (24 hours).

At t=0 and t=24 hours, the capsules were taken out of the incubation medium and examined visually. The results after two hours incubation period in gastric fluid indicate partial dispersion of the oral liquid composition in the medium, whereas the solid composition of WO 2017/098502 remained non-disintegrated. By the end of the twenty-four hours period of the experiment, complete dispersion of the oral liquid composition was observed, while the solid composition of WO 2017/098502 has eroded, but did not disintegrate. However, a few changes in the solid compositions were noted, namely, color change to purple, swelling and softening.

It has also been observed that at the beginning of the incubation period, the capsules settled down at the bottom of the flasks, then after 0.5 h the capsules floated for the next ten hours. At t=24 hours, the dosage forms settled down again.

### CONCLUSION

Based on the above results, a capsule could be designed comprising the cannabinoid novel liquid composition and the cannabinoid composition of WO 2017/098502.

1. Essentially water-free liquid composition for oral administration, comprising:
from 25% to 75% by weight of one or more cannabinoid(s); and from 25% to 59% by weight one or more phospholipid(s); and optionally one or more antioxidant(s).

2. A liquid composition according to claim 1, comprising:
from 25 to 70% by weight of one or more cannabinoid(s);
from 20 to 55% by weight one or more phospholipid(s).

3. A liquid composition according to claim 2, comprising:
from 30 to 50% by weight of one or more cannabinoid(s);
from 30 to 50% by weight one or more phospholipid(s).

4. A liquid composition according to claim 2, comprising not less than 50% by weight of one or more cannabinoid(s).

5. A liquid composition according to claim 1, further comprising one or more glycols.

6. A liquid composition according to claim 1, further comprising one or more of vegetable oils.

7. A liquid composition according to claim 5, wherein the concentration of the glycol(s), vegetable oil(s) or a mixture thereof is not less than 15% by weight.

8. A liquid composition according to claim 1, devoid of glycols, oils or both glycols and oils.

9. An orally administrable liquid composition obtainable by, or obtained by, mixing phospholipids and cannabinoid(s) to form a liquid, characterized in that the mixing takes place in the absence of other ingredients, wherein the optional addition of one or more auxiliary liquids follows after the cannabinoid(s) and phospholipids are associated in a liquid form.

10. A liquid according to claim 1, wherein the one or more cannabinoid(s) constitutes the predominate component relative to the phospholipids.

11. A process for preparing the liquid composition according to claim 1, comprising mixing phospholipids and cannabinoid(s) to form a liquid, characterized in that the mixing takes place in the absence of other ingredients at room temperature, wherein optional addition of auxiliary liquids follows after the cannabinoid(s) and phospholipids are associated in a liquid form.

12. Orally-administrable unit dosage form comprising the composition of claim 1.

13. Orally-administrable unit dosage form of claim 12, which is a liquid-filled capsule, wherein the liquid is the essentially water-free liquid composition comprising from 25% to 75% by weight of one or more cannabinoid(s); and from 25% to 59% by weight one or more phospholipid(s); and optionally one or more antioxidant(s).

14. Liquid-filled capsule according to claim 13, further comprising one or more solid bodies made of phospholipids/cannabinoids solid mixture, wherein said mixture is proportioned in the range of at least 60:40 in favor of the phospholipids.

15. Liquid-filled capsule according to claim 14, wherein the phospholipids/cannabinoids solid mixture is proportioned in the range from 70:30-90:10.

16. Liquid-filled capsule according to claim 14, wherein the one or more solid bodies is/are suspended in the liquid composition.

17. Liquid-filled capsule according to claim 14, comprising an inner capsule loaded with one or more of the solid bodies, wherein said inner capsule is encapsulated in the liquid-filled capsule.

18. A method for treating a disease or a condition in a mammal, combating the progress of a disease, or relieving symptoms associated with a disease, wherein said disease or condition are treatable by cannabinoids, the method comprises orally administering an essentially water-free liquid composition for oral administration comprising from 25% to 75% by weight of one or more cannabinoid(s); and from 25% to 59% by weight one or more phospholipid(s); and optionally one or more antioxidant(s) or the unit dosage of claim 12 to the mammal.

19. A method according to claim 18, for treating pain.
